(19) United States
(12) Patent Application Publication
(10) Pub. No.: US 2011/0189764 A1
(43) Pub. Date: Aug. 4, 2011

(54) SINGLE USE DEVICE INCORPORATING A CONTAINER AND SCAFFOLD

(76) Inventor: Nathan Starbard, Boston, MA (US)

(21) Appl. No.: 13/072,737

(22) Filed: Mar. 27, 2011

(51) Int. Cl. C12M 1/00

(52) U.S. Cl. 435/289.1; 435/283.1; 435/307.1

(57) ABSTRACT

The present invention relates to a single use device, alternatively called an assembly, that primarily comprises a single use container or bioreactor that incorporates a scaffold and is used in the growth, processing and/or preservation of cells, sterile fluid or related biopharmaceutical product and that may be at least partially sterilized by irradiation or exposure to ethylene oxide (ETO).

### TECHNICAL FIELD

The present invention concerns a device incorporating a single use container, assembly or bioreactor that contains a scaffold and is used for cell culturing or the processing, storage or transport of a biopharmaceutical product or related fluid.

### BACKGROUND ART

Biotechnology, pharmaceutical and related industries have historically used stainless steel, or otherwise reusable, processes and devices. Re-useable processes must be cleaned and sterilized between batches. The cleaning and sterilization usually requires the use of steam and/or chemicals to accomplish the task. Additionally, for regulated products such as pharmaceuticals, the sterilization process has to be validated to show that it could repeatedly sterilize the device. The cleaning and sterilization processes and the validation are time consuming and expensive and cannot be varied without a new validation. Re-useable processes also have significantly higher startup costs in material and installation, and often require a much larger footprint.

As a result, in recent years these industries are increasingly moving towards the use of single use (disposable) containers, tubing and ancillary equipment in their research, manufacturing and processing of sterile fluids. The use of disposable devices eliminates or minimizes the need for cleaning and sterilizing equipment between batches. Newly developed bioreactors, for example, which are used in growing cells or microorganisms, commonly comprise a polymeric flexible bag like container. The cells or microorganisms are grown within the polymeric bag. Polymeric tubing coupled with the container is used for adding and removing material from the container. Once a batch is complete the bag and tubing are disposed of and a new bag is used for the next batch. Similar polymeric containers are used in the storage, transportation, processing and freezing of sterile fluids throughout biotechnology and pharmaceutical applications. Such containers are often combined with sterile tubing, connectors, valves, filters, sampling devices and so forth to complete a sterile system that is often called an assembly. Small scale bioreactors or certain containers, such as sampling containers, may additionally be constructed of a rigid plastic, such as polycarbonate, to achieve the same function.

Applications involving cells are sometimes performed in a variety of porous matrices, called scaffolds, which can support cell growth on or within their structures. Scaffolds are used to provide a structure for cells to adhere to and through which media, nutrients and other materials, such as growth factors, can be delivered. Such scaffolds are increasingly used in stem cell and tissue engineering applications. To grow a functional tissue, for example, it is often necessary to culture cells in three dimensions to mimic the natural extra cellular matrix (ECM) of living organisms.

### SUMMARY

Re-usable processing, research, culturing and storage technologies require cleaning and sterilization, increased start-up costs and costly validations. They also create difficulties for technology transfer and can limit scalability and reproducibility.

Current single use assemblies do not offer a mechanism for cell adhesion or cell growth in a three dimensional format which is required for certain applications. Many cells cannot live or remain undifferentiated without being in or attached to a three dimensional structure. Cells also cannot develop into functional tissue without a three dimensional environment. In addition to cell growth, scaffolds can provide benefits when processing, storing or freezing cells and other biopharmaceutical products.

Scaffolds are often manually used in conjunction with tissue flasks or well plates. Those systems do not offer a truly closed, and therefore sterile, environment throughout use. Lack of true sterility will limit their use in any commercialized or clinical product or process. These devices are also limited in size and function and are not able to be scaled up to any appreciable volume. Single use assemblies and containers offer a format compatible with many other solutions to common industry problems such as sterile fluid addition, mixing, sampling, monitoring, filtration and fluid transfer through a variety of products and technologies currently available. These solutions do not generally exist with formats such as tissue flasks or well plates.

The present invention advantageously combines the benefits of single use devices and scaffolds. By incorporating a scaffold into a single use bioreactor, container or assembly and sterilizing all or part of the closed system the user can safely culture, transport, process, freeze, sample and store the fluid and/or product without the risk of contamination and also thereby reducing the cleaning and/or validation costs associated with conventional methods. Technology transfer, the process of moving, transferring or duplicating processes in different locations, is also made significantly easier through the use of such a device.

### BRIEF DESCRIPTION OF THE DRAWINGS

FIG. 1 is a single use, flexible disposable container made of joined film layers that has a scaffold inside of the walls of the container. The scaffold drawn in FIG. 1 is for representation only. Many details of scaffolds are only visible under intense magnification. The size of the scaffold is variable based on type or application.

FIG. 2(A-C) shows three representative drawings of scaffold details. Details may only be visible under magnification while other times details may be visible to the eye. There are many other configurations of scaffolds with various structures.

FIG. 3 shows a single use assembly comprising two flexible containers connected by way of tubing and a single use filtration device. One container is pre-filled with liquid media. A rigid container is connected to the flexible container. A representative scaffold is shown in one flexible container, although a scaffold may be present in any combination of one, two or all three of the containers.

FIG. 4 shows a rigid single use bioreactor with three small sampling containers and one large flexible container connected to the bioreactor. A scaffold is depicted within the bioreactor although a scaffold may be present in one or more of any of the five containers shown in FIG. 4.

FIG. 5 shows a large three-dimensional flexible bioreactor or container. Separate male and female sterile tube to tube connectors would connect the container to a point in the manufacturing process depicted by the broken end of the tubing.

FIG. 6 shows a series of three flexible sampling containers, with scaffolds, attached to a stainless steel processing tank or bioreactor by way of a sterile connector and tubing.

### DESCRIPTION OF THE EMBODIMENTS

With reference to the figures, a single use device comprising a container 1, 12, 15, 16, 17 with a scaffold 7 or scaffold containing or producing substance that is internal to the container, tubing ports 3, 8 and associated tubing 2 as well as ancillary equipment such as connectors 4, 6, 13, 18, 20, filters 5, 9, 11, valves 21, clamps 14 and so forth is described. All or a portion of the device is sterilized by irradiation or exposure to ethylene oxide (ETO).

The single use container 1, 12, 15, 16, 17 of this invention is formed of a polymeric composition such as polyethylene (including ultrahigh molecular weight polyethylene, linear low density polyethylene, ultralow, low or medium density polyethylene); polypropylene (PP); polyamide (PA); polyethylene terephthalate (PET); polysulfone (PS); polyethersulfone (PES); ethylene vinyl alcohol (EVOH); polyvinyl chloride (PVC); polyvinyl acetate (PVA); ethylene vinyl acetate (EVA); ethylene vinyl acetate copolymers; polycarbonate (PC); multilayered laminates of different thermoplastics; as well as other polymers and plastics (including thermoplastic polymers, homopolymers, copolymers, block copolymers, graft copolymers, random copolymers, alternative copolymers, terpolymers, metallocene polymers) and derivatives thereof. Such materials are available from a wide range of chemical manufacturers. Single use containers used in the biotechnology and pharmaceutical markets are available from a variety of suppliers such as Sartorius Stedim Biotech SA of France; ThermoFisher Scientific Inc. (formerly HyClone) of Logan, Utah; Millipore Corp. of Billerica, Mass.; Advanced Scientifics Inc. of Millersburg, Pa.; Pall Corp. of Port Washington, N.Y. and so forth. These containers generally range in size from 20 milliliters to 2000 liters or more. Other materials used in the present invention to make the container can be those typically used in the pharmaceutical and biotechnology industries for disposable bioreactors, fermenters, containers and the like and includes specialty or proprietary polymers, an example of which is the HyQ CX5-14 film available from ThermoFisher Scientific which is coextruded multilayer film with an outer layer of elastomer with an EVOH barrier layer and an ultra low density polyethylene (ULDPE) product contact layer.

Incorporated into the container 1, 12, 15, 16, 17 are one or more tube ports 3, 8 which are in fluid communication with the chamber. One, two, three or more tube ports can be present depending on the intended use of the container. Tube ports may be individually attached to the container, or as several attached to a manifold or other such device that is connected to the container. Tube ports attached to the face of the film are often called face ports 8. Each tube port can serve a different purpose depending on the type of processing or application to be undertaken. In addition, such as when the container is used as a bioreactor 15 for growing cells or microorganisms, tube ports 3 can be used to provide various probes such as temperature probes, pH probes, dissolved oxygen probes and the like, access to the chamber. Additional components such as filters 5, 9, 11, connectors 4, 6, 13, 18, 20, valves 21, sampling devices and so forth may also be incorporated either directly onto the container or attached to the container through tubing 2 and/or tube ports 3, 8 attached to the container. Tubing 2 used in the invention may incorporate a method of detachment 10 such as crimping, cutting, folding and so forth such that the container, tubing or affiliated device, such as a filter, may be removed from the container or such that the container can be removed from the process or remainder of the overall assembly.

The scaffold 7 used in the invention may be formed from any suitable material. Suitable materials include polymers and plastics (including thermoplastic polymers, homopolymers, copolymers, block copolymers, graft copolymers, random copolymers, alternative copolymers, terpolymers, metallocene polymers, polyethylene (PE), polypropylene (PP), polyvinyl chloride (PVC), polycarbonate (PC), polyethylene terephtalate (PET), polyethersulfone (PES), polysulfone (PS), polyetheretherketone (PEEK), polytetrafluoroethylene (PTFE), polyurethane (PU), polycaprolactone (PCL), polyglycolic acid (PGA), poly(lactic-co-glycolic acid) (PLGA), poly(lactic acid) (PLA), poly(L-lactic acid) (PLLA), and derivatives thereof); fabrics and fibers (including woven and nonwoven fabric, spunbonded fibers, meltblown fibers, nanofibers, polycellulose fibers, polyester fibers, polyurethane fibers, polyolefin fibers, polyamide fibers, cotton fibers, copolyester fibers, and silk); metals (including sintered metals); fiberglass; glass; hydrogels (including those formed of peptides); films; fibrin; hydroxyapatite or tricalcium phosphate; aliginate; calcium carbonate; calcium phosphate; cellulose; ceramics (including ceramic glasses); chitin; collagen (including collagen of various types); glycoaminoglycans; carbon nanotubes; demineralized bone matrix; decellularized tissue; and combinations or mixtures thereof. The scaffold may be one piece, several pieces or a multitude of small pieces, such as when the scaffold is a plurality of microspheres.

A subset of scaffolds is microspheres. Microspheres are small, usually rounded, individual particles that also create a three dimensional structure and provide a method for delivery of bioactive molecules and can facilitate cell growth and differentiation. They can also deliver stem cells to a targeted area for patient treatment. Microspheres are used in plurality to create the necessary environment. Microspheres can be used by themselves, or also in conjunction with another separate scaffold. The present invention includes when microspheres are incorporated within the single use container or used as the scaffold.

A flexible polymeric single use container 1, 16, 17 is generally made by sealing the edges of its film layers. Tube ports or manifolds of tube ports may be joined to the seams of flexible containers. Ports may also be welded to the face of the polymer film. A rigid plastic single use container 12, 15 is made by any common plastic molding and/or welding technique used in the creation of plastic containers. Tube and access ports are welded to or molded into the container.

Tubing is attached to the tube ports of the container. Other accessories and components such as filters, connectors, probes and mixers may be attached to the tubing, a tube or access port, directly to the container or incorporated within the container such that the component or accessory may be located inside of the container.

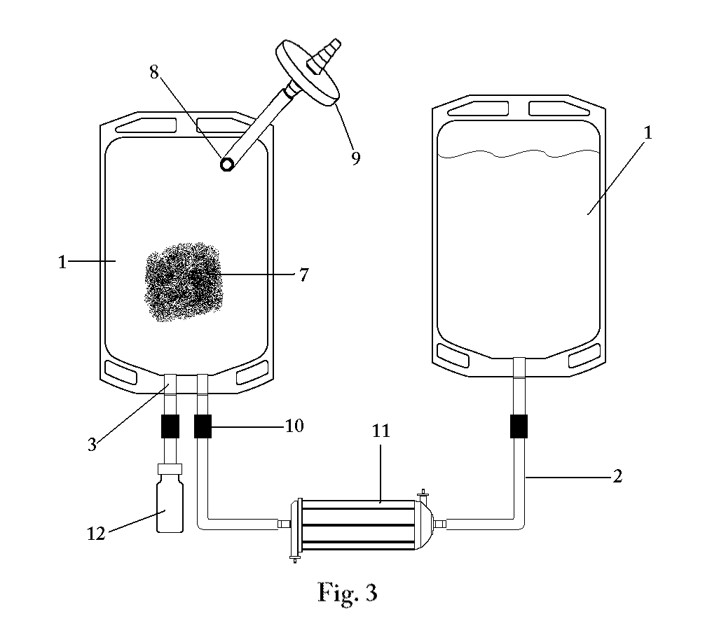

As scaffolds 7, FIG. 2 can be any suitable three dimensional material used for cell adhesion they can be produced by nearly any manufacturing technique. Less advanced techniques can include welding, cutting, molding and so forth. More advanced techniques of manufacturing scaffolds can include being made by self assembly, phase separation, freeze drying, electro-spinning, surface selective laser sintering and so forth. The scaffolds may be modified with bioactive molecules such as growth factors, drugs, adhesion peptides and so forth. The composition of a scaffold can be used to persuade stem cells to differentiate in a specific manner. Scaffolds may be both biodegradable and not biodegradable and may or may not be directly implanted into a patient for certain tissue engineering applications. Scaffolds may be pre-wetted with culture media. This can involve the application of pressure or vacuum to the container of the device. Scaffolds may also be exposed to solutions, such as alcohol, to increase wet-ability. Mixing of the container may facilitate more efficient cell seeding on the scaffold.

A scaffold is incorporated into one or more of the containers of the device such that the scaffold is located within the walls of the container. This will usually occur directly, when the scaffold is placed inside of a container that is part of the device. The scaffold may be placed within the film layers of a flexible container prior to seaming. The scaffold may be placed inside of a rigid plastic container prior to final assembly or welding. The scaffold may be placed within a fully constructed container through a tube or face port or other access point. Final assembly of the device should occur in a clean room or similar environment to minimize particulates and contaminants.

The present invention is most commonly sterilized by exposure to irradiation. The two forms of irradiation most commonly used in the pharmaceutical and biotechnology industries for sterilization are exposure to gamma rays and cathode rays, also called electron beams or e-beams. X-rays and ultraviolet (UV) light are other sources of radiation and may also be used in some circumstances. Exposure to ETO may also be used for sterilization. The present invention may be sterilized through a combination of irradiation, exposure to ETO and other techniques such as autoclaving. For example, a filter or tubing assembly may be autoclaved and attached by a sterile connector to a single use container with scaffold that has been gamma irradiated. Another example would be a scaffold sterilized by exposure to ETO and incorporated into a gamma irradiated assembly or bioreactor in a sterile environment, such as a hood.

Materials have different levels of compatibility with respect to exposure to irradiation. Many materials turn brittle, discolor or crack following exposure to irradiation used for sterilization. Materials may be altered or blended to increase the contact dosage able to be applied. An irradiation compatible material is one that is able to perform its intended function following sterilization by irradiation. As the intended function may differ, a material may be considered compatible for one application of the device, while not compatible for another application. Furthermore, some materials may be more or less affected by one form of irradiation used for sterilization than another.

Liquid media used in cell culturing or processing may be pre-packaged in irradiated sterile containers. The containers are connected by way of a sterile connector, single use filtration device or both to a process, tank or assembly. A prefilled media container may be used in conjunction with a container that has a scaffold in it as in FIG. 3. The pre-filled container may also include a scaffold.

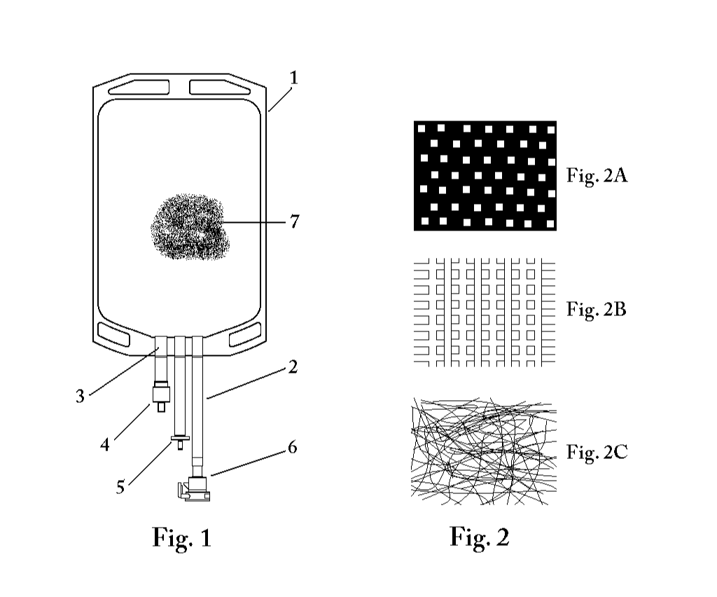

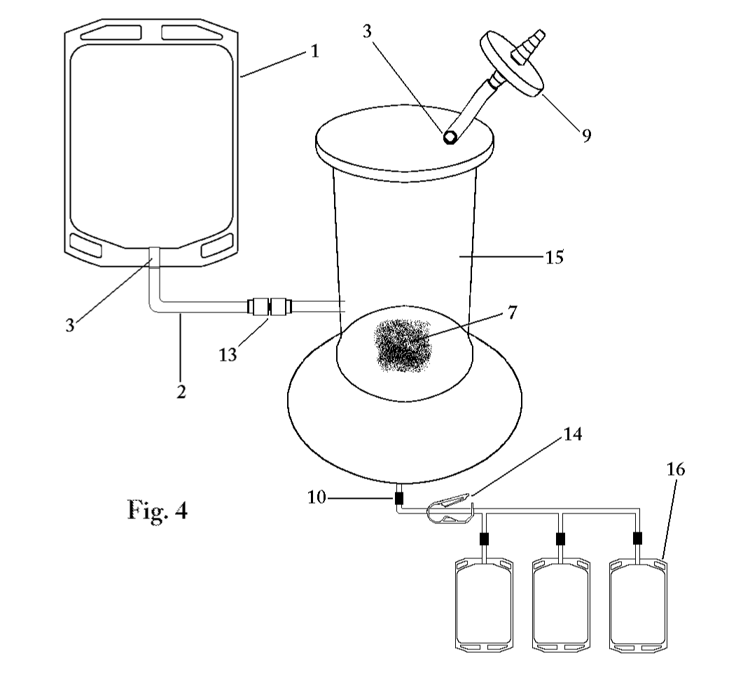

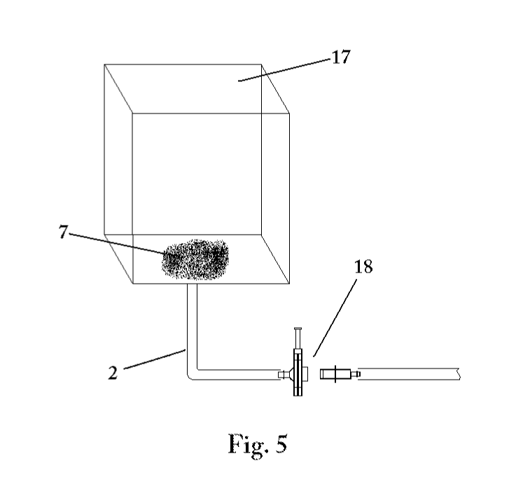

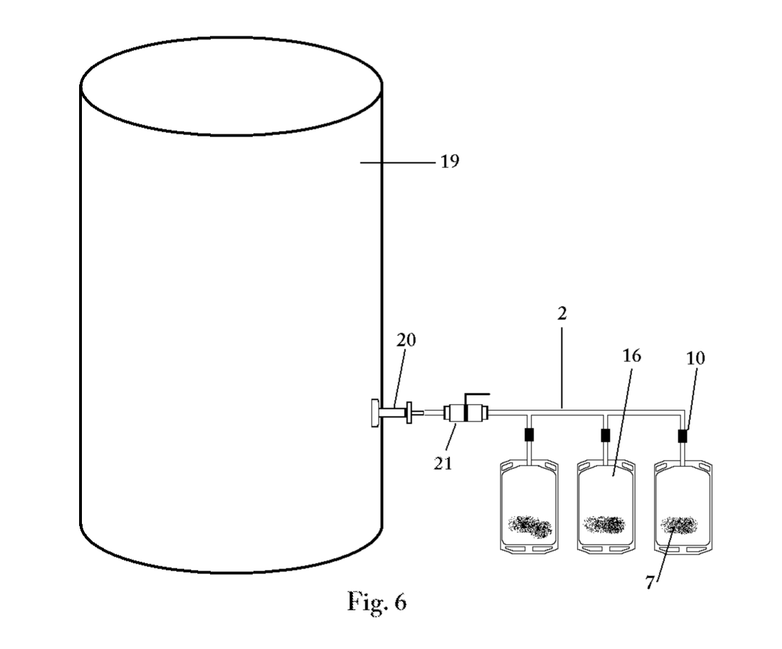

Large flexible single use containers or bioreactors 17 used in the invention are often placed inside of a re-useable container, such as a stainless steel or plastic bin or tote, for support. Because the re-useable bin or tote is not in fluid contact it is not a critical component of the process. Sterile tube welders may be used to combine two or more pieces of a single use assembly or to attach an assembly to the process. The invention may be used independently of any process as in FIG. 1, as an entire disposable process in and of itself as in FIG. 4, or attached to a process such as when connected to a re-useable tank 19 as in FIG. 6, bioreactor or other processing equipment.

The single use containers of the invention may be wirelessly enabled. The wireless communications device may be a RFID tag having a communication and storage or memory component or other wireless device such as Bluetooth or Zigbee wireless enabled communications devices. By wirelessly enabling the device one can track the device history or important information such as manufacture date, lot number, shelf life, sterilization date and the like.

The invention may be assembled either entirely or partially by the end-user of the device, the device or a device component vendor, a contract manufacturing organization, a third party sterilizer or other company providing a service related to the device or a combination thereof.

### REFERENCE SIGNS LIST

1 Single use flexible (bag) container

2 Tubing

3 Tube port

4 Tube connector

5 Syringe type vent filter

6 Colder type connector

7 Representative drawing of scaffold

8 Face welded tube port

9 Vent filter

10 Crimp or cut tubing disconnect

11 Single use capsule filter

12 Rigid plastic sample container or freezing vial

13 Luer type connection

14 Tubing clamp

15 Rigid single use bioreactor

16 Flexible single use sample container

17 Large three-dimensional flexible container

18 Male and female ends, sterile fluid connector

19 Stainless process tank or bioreactor

20 Sterile connector, tri-clamp to tubing

21 Tubing valve

1. A device comprised of one or more single use containers;
wherein one or more of the containers incorporates a scaffold, and the containers have one or more tube or face ports; and
wherein at least a portion of the device is sterilized by irradiation or exposure to ethylene oxide (ETO); and
wherein the device is used for cell culturing, research or for fluid processing, storage, sampling, freezing or transportation in the pharmaceutical, biopharmaceutical, biotechnology or related industries.

2. The device of claim 1 wherein one or more containers are configured as flexible (bag) or rigid containers or a combination thereof.

3. The device of claim 1 wherein one or more containers are used as a bioreactor.

4. The device of claim 1 wherein the scaffold is transferred to the device's container prior to device usage.

5. The device of claim 1 wherein the scaffold is incorporated into the device during device or container manufacturing or assembly.

6. The device of claim 1 wherein said device is attached to a tank, storage vessel or containment structure of any type regardless of whether said vessel or structure is, in itself, part of a single use assembly or device.

7. The device of claim 1 wherein said device incorporates a connector or filter designed to maintain the sterility of said device or its related process, product or associated fluid stream.

8. The device of claim 1 wherein one or more containers incorporates a mixing technology.

9. The device of claim 1 wherein said device incorporates a method of cutting, crimping or otherwise disconnecting the device's tubing so as to maintain the sterility of a fluid, a container or the device.

10. The device of claim 1 wherein the device incorporates a method of fluid control such as a valve or clamp.

11. The device of claim 1 wherein one or more of the containers are wirelessly enabled.

12. The device of claim 1 wherein the container or scaffold is filled or incorporated with cell media, feeder cells, growth factors or other materials designed to grow, store, freeze, preserve or induce differentiation in cell populations.

13. The device of claim 1 wherein the scaffold is or is not biodegradable.

14. The device of claim 1 wherein the scaffold is considered a plurality of microspheres.
